(19) United States
(12) Patent Application Publication
(10) Pub. No.: US 2021/0393573 A1
(43) Pub. Date: Dec. 23, 2021

(54) TABLETS, FORMULATIONS AND METHODS FOR LOW MELTING POINT ACTIVE INGREDIENTS

(71) Applicant: KELSIE BIOTECH, LLC, Boulder, CO (US)

(72) Inventors: Lia REBITS, Lakewood, CO (US); Xuno GILDELAMADRID, Fort Morgan, CO (US)

(21) Appl. No.: 17/287,361

(22) PCT Filed: Oct. 29, 2019
(86) PCT No.: PCT/US19/58537

§ 371 Date: Apr. 21, 2021

Related U.S. Application Data
(60) Provisional application No. 62/752,719, filed on Oct. 30, 2018.

(51) Int. Cl. A61K 31/352; A61K 9/20; A61K 31/05

(52) U.S. Cl. CPC A61K 31/352; A61K 9/2054; A61K 9/2009; A61K 9/2095; A61K 9/2018; A61K 31/05; A61K 9/2013

(57) ABSTRACT

A tablet comprises a granulate of an active pharmaceutical ingredient comprising at least one cannabinoid having a melting point less than about 80° C.; sugar, sugar alcohol, or a combination thereof; microcrystalline cellulose having an average particle size less than about 25 μm; silica, silicified microcrystalline cellulose, or a combination thereof; and lubricant comprising sodium stearyl fumarate and lecithin. Methods of forming such a tablet using direct compression of a tablet formulation can be conducted on a large manufacturing scale.

### FIELD OF THE INVENTION

The present invention is directed to tablets, tablet formulations and tableting methods for active pharmaceutical ingredients having a relatively low melting point. In specific embodiments, the active pharmaceutical ingredient comprises at least one cannabinoid having a relatively low melting point. The tablets, tablet formulations and tableting methods allow efficient large-scale tablet formation by direct compression.

### BACKGROUND OF THE INVENTION

Successful formulation and large-scale manufacturing of an active pharmaceutical ingredient (API) into a tablet form requires simultaneous optimization of a multitude of variables. In addition to tailoring properties to meet patient-interface demands such as taste, texture, and disintegration time, variables that affect efficiency of production must also be considered. A number of bulk powder properties including density, flowability, compressibility, and stickiness can have a significant effect on the suitability of the powder for direct compression, and lack of optimization of any of these variables can interfere with and stymie large-scale tablet production development. Although formulation deficiencies can sometimes be compensated for through the adjustment of equipment and/or manufacturing parameters, i.e., through the application of increased pressure, lower throughput, etc., an optimized formulation is one that allows efficient and uninterrupted production while causing the least amount of stress on the production equipment.

Direct compression is an efficient and economical process for forming pharmaceutical dosages in the form of tablets from powder formulations. Generally, when a powder formulation is subjected to tableting by direct compression, energy is transferred from the tableting press to the powder formulation. Low melting point APIs which are liquid at room temperature or APIs which melt at temperatures encountered in such tableting operations are problematic in processes for forming tablets in that such APIs are poorly compressible and can interfere with the formation of particle interconnectedness within the tablet during compression. This interference may cause some powder particles to adhere to the tablet press punch rather than to the formed tablet. Adherence of powder particles to the punch eventually leads to picking (sticking to engraved areas of the punch face), sticking, and capping of the tablets, poor product quality, production inefficiency, stress on the equipment, and interruptions in manufacturing for equipment cleaning. Problems created by powder particle sticking in small scale or pilot manufacturing are often magnified when a production process transitions to large scale production.

Resolution of the problems created by powder particle sticking during direct compression tableting in the pharmaceutical industry in general can be challenging in view of the particular characteristics of the API and excipient matrix. Components of the formulation that are included in order to satisfy certain requirements, such as flowability requirements of specific equipment, fast disintegration, API stability, etc., may themselves contribute to other issues such as sticking, and solving one formulation problem can therefore often lead to the creation of others. As such, formulations that have been optimized to resist sticking during tablet manufacture are often utilized only for a specific API, excipient or combinations thereof, or particular characteristics thereof, and are not universal across all pharmaceutical formulations. Successful formulation efforts take into account the interaction between the physical and/or molecular properties of the API, the excipients needed to produce a specified form and administration properties, and the requirements of particular processing equipment, and result in a formulation that has been precisely engineered and is unique for the application.

APIs extracted from the Cannabis plant, commonly referenced as cannabinoids, have been disclosed for various medical uses, including, but not limited to, treatment of epileptic seizures, treatment of cancer, reducing adverse effects of cancer treatments, pain management, and treatment of auto-immune disorders. Accordingly, dosage forms which facilitate use of cannabinoids for such therapeutic treatments are desired, particularly dosage forms which can be efficiently manufactured on a large scale. Generally, cannabinoids are difficult to incorporate into tablets formed by direct compression of powder formulations primarily due to their low melting points. Many cannabinoids, such as Δ9-tetrahydrocannabinol (THC), have melting points around room temperature and present as viscous, resinous oils that are extremely sticky to the touch and exhibit poor compressibility. Even those cannabinoids that are crystalline solids at room temperature, such as cannabidiol (CBD), often have melting points just above room temperature (69-70° C. for CBD), and therefore are susceptible to melting during the compression process used for tableting and interfering with process efficiency, particularly in large scale manufacturing.

Accordingly, a need exists for the ability to formulate low melting point APIs such as cannabinoids in powder formulations which allow efficient tableting, for example, by direct compression, particularly on a large scale manufacturing basis.

### SUMMARY OF THE INVENTION

Accordingly, it is an object of the invention to provide tablets containing low melting point APIs, such as, but not limited to, one or more cannabinoids, which may be easily and efficiently formed by direct compression, and, in certain embodiments, on a large scale manufacturing basis.

In one embodiment, the invention is directed to a tablet comprising a granulate of an active pharmaceutical ingredient having a melting point less than about 80° C.; sugar, sugar alcohol, or a combination thereof; microcrystalline cellulose having an average particle size less than about 25 μm; silica, silicified microcrystalline cellulose, or a combination thereof; and lubricant comprising sodium stearyl fumarate and lecithin. In a specific embodiment, the active pharmaceutical ingredient comprises at least one cannabinoid.

In another embodiment, the invention is directed to a method of forming a tablet containing an active pharmaceutical ingredients having a melting point less than about 80° C. The method comprises granulating a mixture of the active pharmaceutical ingredient and sugar, sugar alcohol, or a combination thereof, mixing the resulting granulate with (a) additional sugar, sugar alcohol, or a combination thereof, (b) microcrystalline cellulose having an average particle size less than about 25 μm, (c) silica, silicified microcrystalline cellulose, or a combination thereof, and (d) lubricant comprising sodium stearyl fumarate and lecithin to provide a tablet formulation, and direct compressing the resulting mixture to form a tablet. In a specific embodiment, the active pharmaceutical ingredient comprises one or more cannabinoids.

The tablets, the formulations for forming tablets, and the methods of the invention are advantageous in allowing efficient production of tablet dosage forms containing an API having a low melting point from a powder using direct compression tableting processes and equipment. Additional objects and advantages of the invention will be more fully apparent in view of the following detailed description.

### BRIEF DESCRIPTION OF THE DRAWINGS

Various aspects of the invention will be more fully understood in view of the drawings, in which:

FIG. 1A shows a schematic diagram of a compressed powder of mannitol, and FIG. 1B shows a schematic diagram of a compressed powder of mannitol and the very fine particle microcrystalline cellulose employed according to the present invention.

FIG. 2 shows comparative tablets as described in Comparative Example 2.

FIG. 3 shows inventive tablets as described in Example 3.

FIG. 4 shows inventive tablets as described in Example 4.

FIGS. 5A-5D show comparative tablets and punch surfaces from direct compression processes as described in Comparative Example 5.

These drawings are illustrative of certain aspects and embodiments of the invention and are not limiting of the invention as defined by the claims.

### DETAILED DESCRIPTION

The invention provides tablets containing one or more low melting point APIs. The tablets comprise a unique combination of ingredients which allows tablet manufacture by direct compression while avoiding manufacturing problems of the prior art typically encountered with direct compression tablet formation of powder formulations containing a low melting point API. Thus, in certain embodiments, the tablets and methods of the invention allow efficient tablet manufacturing on a large scale manufacturing basis.

Specifically, the tablets and methods of the invention employ an API having a melting point less than about 80° C. Within the present disclosure, reference to an API having a melting point less than about 80° C. encompasses any of (i) a single such API having a melting point less than about 80° C., (ii) a combination of two or more of such APIs having a melting point less than about 80° C., and (iii) a combination of one or more of such APIs having a melting point less than about 80° C. with one or more APIs which have a melting point greater than 80° C. In specific embodiments, the API having a melting point less than about 80° C. comprises at least one cannabinoid. In another specific embodiment, the API having a melting point less than about 80° C. comprises two or more cannabinoids. In additional embodiments, the API having a melting point less than about 80° C. comprises CBD, THC, tetrahydrocannabinolic acid (THCA), cannabidiolic acid (CBDA), cannabinol (CBN), cannabigerol (CBG), cannabichromene (CBC), cannabitriol (CBT), or combinations of two or more thereof. To the extent that any one of these cannabinoids has a melting point greater than about 80° C., they may advantageously be combined with one or more additional cannabinoids having a melting point less than about 80° C. in compositions according to the invention. In additional embodiments, the API having a melting point less than about 80° C. comprises CBD, THC, or both CBD and THC.

The tablets of the invention may include the API or a combination thereof in any desired amount. In a specific embodiment, the tablets contain from about 0.1 to about 20 wt % API or a combination of APIs, or, more specifically, from about 0.1 to about 20 wt % cannabinoid, wherein the cannabinoid may be a single cannabinoid or two or more cannabinoids. In a more specific embodiment, the tablets contain from about 0.2 to about 10 wt % API or a combination of APIs, or, more specifically, from about 0.2 to about 10 wt % cannabinoid, wherein the cannabinoid may be a single cannabinoid or two or more cannabinoids. In other embodiments, the tablets may contain greater than 20 wt % API or combinations of APIs, or, for example, up to about 30 or 40 wt % API or combination of APIs, or, in each embodiment, cannabinoid, wherein the cannabinoid may be a single cannabinoid or two or more cannabinoids.

In order to provide a powder formulation containing the API having a melting point less than about 80° C. that can be tableted using direct compression in an efficient manner, the API having a melting point less than about 80° C. is added to the powder formulation in the form of a granulate. To avoid tableting problems owing to the sticky nature of an API having a melting point less than about 80° C., such as the aforementioned cannabinoids, especially those that are not solid at room temperature, the API is incorporated into an excipient matrix by granulation before mixing with a bulk powder that will be used for direct compression into tablets. Generally, producing tablets by the direct compression method requires physical mixing of the API and excipients, i.e., in a tumbler mixer, followed by placement of the resulting bulk powder into the tableting press, and direct compression. In contrast, preparation of tableting powders by granulation requires that the API and one or more excipients are first combined together into granular particles that are a composite of API and excipient. The granular particles are then mixed with remaining tablet excipients to provide a formulation for direct compression tableting. Incorporation of the poorly-compressible low melting point API, such as a cannabinoid, into a granule that can then be mixed immediately into a direct compression mixture improves the tableting process.

In one embodiment, the API is mixed with at least a portion of one or more excipients, for example, a portion of the sugar and/or sugar alcohol, to form the desired granulate. The granulate may be formed by any granulation method known in the art. For example, in one embodiment, the API and sugar and/or sugar alcohol, or other excipient, are subjected to wet granulation which involves dissolving the API in a solvent, mixing the solution with an excipient, and removing the solvent. Wet granulation may be conducted in a fluidized bed to form the granulate. In another embodiment, the API and excipient, for example sugar and/or sugar alcohol, are melt granulated. By gently mixing a liquid API, which may first require melting the API, such as a cannabinoid, into a slurry with one or more excipients such as a sugar and/or sugar alcohol, while maintaining the particle matrix of the excipient, the cannabinoid or other API is melted into the excipient particle matrix, creating a fusion particle that reduces the stickiness associated with the pure API, i.e., a pure cannabinoid.

The resulting excipient-API composite granulates will typically have a size in a range of from about 65 μm up to about several mm. The granules may then be ground into a fine particle size, for example, in a range of about 10 μm to about 50 μm, and the resultant fine powder is then tumbled with the rest of the excipients to form a bulk powder for direct compression. The present method is therefore a combination of direct compression tableting in which most of the excipients and the API are physically mixed by tumbling, but the API is first processed by granulation into a more easily compressible form.

Melt granulation is advantageous in that a solvent as is employed in wet granulation is not required. The advantage of a solvent-free melt granulation method is that the composite powder is ready for grinding and incorporation into the bulk tableting powder after only a short period of cooling, whereas complete removal of solvent must be accomplished for solvent-based wet granulation methods. Removal of solvent is either extremely time-consuming if allowed to air dry, or requires vacuum-supplied reduced pressure, which necessitates additional expensive equipment and energy. Additionally, residual solvent levels in the dried powder must be monitored, and inadequate solvent/moisture removal may lead to batch-to-batch inconsistencies. If the residual solvent/moisture level in the tableting powder is too high, sticking will result, which leads to product storage instability and manufacturing inefficiencies.

In addition to the API-excipient fine powder produced from granulates, the tablets of the invention comprise sugar and/or sugar alcohol, which serves as a bulk tableting powder. Suitable sugars and sugar alcohols include, but are not limited to, fructose, galactose, glucose, lactose, maltose, sucrose, arabinose, dextrose, fucose, mannose, ribose, rhamnose, trehalose, xylose, mannitol, sorbitol, xylitol, isomalt, arabitol, ribitol, galactitol, pharmaceutically acceptable inositol such as myo-inositol, maltitol, lactitol, iditol, and fucitol. Any combination of two or more of these sugars and/or sugar alcohols may be employed. As noted, a portion of the sugar and/or sugar alcohol may be used to form the API-containing granulate. In a specific embodiment, about 1 to about 50 wt % of the sugar and/or sugar alcohol included in the tablet may be used in the granulation step, with the remaining amount of sugar and/or sugar alcohol being added as a tableting excipient to the bulk powder with which the API-excipient fine powder is mixed to form a bulk powder formulation for compression. The sugar and/or sugar alcohol provides excellent flowability characteristics to the powder formulation. In a specific embodiment, mannitol is employed as the sugar alcohol. The very low hygroscopicity of mannitol contributes to elongated shelf life of the tablet product. Mannitol also provides excellent disintegration properties, mouthfeel, and a favorable taste profile, and lacks cariogenic (tooth decaying) properties.

The sugar and/or sugar alcohol which is added directly to the bulk powder, without granulation, generally will have average particle sizes of about 100 μm or greater, or of about 200 μm or greater, or of from about 300 μm to about 400 μm. In a specific embodiment, mannitol having an average particle size of about of from about 300 μm to about 400 μm is employed as the tableting excipient. In specific embodiments, the sugar and/or sugar alcohol, or, specifically, mannitol, both that used for granulation and that added directly to the bulk powder, is included in the tablets in an amount of from about 45 to about 80 wt %. In a more specific embodiment, the sugar and/or sugar alcohol is included in the tablets in an amount of from about 55 to about 75 wt %. The sugar and/or sugar alcohol used for granulation may be the same or different from the sugar and/or sugar alcohol used as the tableting excipient.

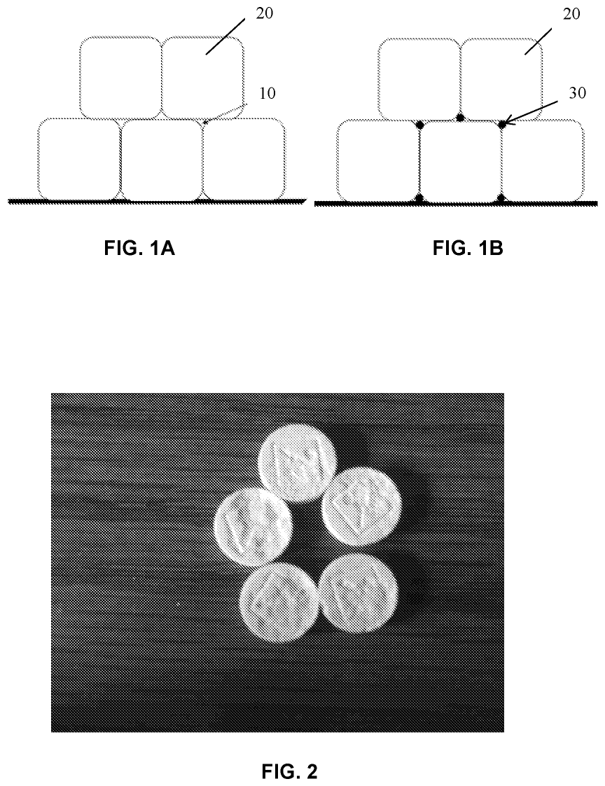

While granulating a low melting point API with an excipient prior to introduction into a bulk powder for tablet formation by compression significantly reduces sticking during the compression process, some sticking is still observed in formulations employing conventional tableting excipients such as microcrystalline cellulose, especially after long production runs used in large scale manufacturing and when using tableting formulations that contain higher percentages of a low melting point API such as a cannabinoid. The present inventors discovered that residual sticking can be further significantly reduced by replacing conventional microcrystalline cellulose such as Avicel® PH 102, which is a standard, commonly-used tableting excipient and has a particle size of about 100 μm or greater, with a combination of (i) very small, fine particle microcrystalline cellulose (MCC) having an average particle size of less than about 25 μm, and (ii) silica or silicified microcrystalline cellulose (SMCC), or a combination of silica and SMCC. The addition of fine particles of MCC allows for strong bridges to be formed between the much larger particles of tableting sugar and/or sugar alcohol under less tableting pressure. Tableting sugar and/or sugar alcohol, i.e., that added directly to the bulk powder without granulation, comprises a majority of the formulation based on the advantageous combination of properties discussed above. However, the relatively large size of the sugar and/or sugar alcohol particles causes difficulties in compressing the bulk powder to tablet form due to the known tendency of sugar and/or sugar alcohol to stick to tableting punches and the significant amount of pressure that needs to be applied to overcome the large dead volume space between the big particles in order for a compact tablet to be formed. Coating these large sugar and/or sugar alcohol particles with a dusting of very fine MCC, which has excellent plastic compressibility characteristics and lacks stickiness, allows some of the large dead volume space to be filled with a material that serves as an effective glue to bind the tablet together but does not stick to the punch. Because the inter-tablet connections are stronger, a more robust tablet is created, and the bulk powder has much less propensity to stick to the tablet press punch. FIG. 1A schematically shows the dead space areas 10 between large sugar and/or sugar alcohol particles 20, for example, mannitol particles, in a compressed powder formulation, while FIG. 1B schematically shows how the dead space areas are filled with the very small size MCC particles 30 between large particles 20 in a compressed powder formulation according to the invention.

In a specific embodiment, the very fine sized MCC is included in the tablets in an amount of from about 5 to about 25 wt %. In more specific embodiments, the very fine sized MCC is included in the tablets in an amount of from about 5 to about 20 wt %.

Additionally, silica and/or silicified microcrystalline cellulose (SMCC), which provides silica, further reduces the sticking tendency of the bulk powder formulation. Silica serves as an adsorbent for any residual moisture that may be introduced to the bulk powder due to high humidity, incompletely dry equipment, etc., without creating an imbalance in other desirable excipient properties. The inventors discovered that with a combination of the very fine sized MCC and the silica and/or SMCC in the inventive powders, sticking of the inventive powder formulations could be entirely reduced to undetectable levels during tableting, even in long manufacturing runs for large scale production. In a specific embodiment, the silica, SMCC, or a combination thereof, is included in the tablets in an amount of from about 1 to about 20 wt %. In more specific embodiments, the silica, SMCC, or a combination thereof, is included in the tablets in an amount of from about 1 to about 15 wt %. In certain embodiments, a weight ratio of very fine particle MCC to silica and/or SMCC of at least 1 is employed. In a specific embodiment, the silica and/or SMCC has a particle size of greater than about 100 μm.

Finally, the tablets of the invention include a lubricant to further optimize the tableting procedure. While magnesium stearate is a well-known lubricant for tableting formulations, the present inventors found that formulations including magnesium stearate exhibited a significant propensity to stick to the punch during direct compression, preventing efficient manufacturing and requiring interruptions for punch cleaning. On the other hand, the inventors discovered that a combination of sodium stearyl fumarate and lecithin provides a bulk powder formulation which, in combination with the previously discussed ingredients, eliminates particle sticking to the punch during direct compression and allows efficient manufacturing, even during large scale manufacturing.

The combination of sodium stearyl fumarate and natural lecithin maximizes the lubrication of the formula and reduces sticking. Lecithin is a natural lubricant due to its surfactant properties, but is only rarely incorporated into tablet formulations. The addition of lecithin to the formulation may increase bioavailability of a cannabinoid as some studies have suggested that the concomitant ingestion of lecithin and cannabinoid may increase bioavailability of the cannabinoid. The lecithin may be derived from a plant source, such as soy lecithin, or from an animal source, such as sheep. The sodium stearyl fumarate provides lubrication of the powder particles through delamination of the plate-like structure of the lubricant particle. Flakes of these lubricants coat each bulk tablet powder particle and further delaminate on contact with the tablet punch, reducing the tendency of the formulation to adhere to the punch surface. Lecithin functions through the reduction of attraction based on polarity of the formulation to the punch by providing regions of hydrophobicity. By changing the polarity of some of the surface molecules, the relatively polar overall formulation presents reduced affinity for the metal of the punch surface, and, consequently, less sticking. Omission of the lecithin from the formulation, or replacement of the sodium stearyl fumarate with more traditional magnesium stearate results in formulations with a much greater propensity to stick to the punch.

In a specific embodiment, the tablets comprise a lubricant of sodium stearyl fumarate and lecithin combined in an amount of from about 0.5 to about 5 wt %. In more specific embodiments, the tablets comprise a combination of sodium stearyl fumarate and lecithin in an amount of from about 1 to about 4 wt %. In additional embodiments, the lubricant comprises at least 50 wt % sodium stearyl fumarate, based on the combined weight of sodium stearyl fumarate and lecithin.

The tablets of the invention may be provided with one or more additional conventional tableting excipients, as long as such conventional excipients do not interfere with the ability of the powder formulations to be efficiently formed by direct compression tableting as discussed herein. Examples include crospovidone, which acts as a disintegrant, flavoring agents, coloring agents, preservatives, other benefit-providing additives such as melatonin, caffeine, GABA (gamma-aminobutyric acid) or other amino acids, and the like. Similarly, the tablets may be provided with one or more coatings as desired subsequent to the tableting procedure.

In a specific embodiment, the tablets include a disintegrant such as crospovidone and are quickly dissolvable upon oral administration. For example, the disintegrant is included in an amount sufficient to dissolve the tablet in a period of from about 10 seconds to about 3 minutes when administered by mouth, for example, as a sublingual tablet. In a specific embodiment, the tablets include from about 1 to about 20 wt %, more specifically, from about 1 to about 10 wt %, of a disintegrant, and in further embodiments, the disintegrant is crospovidone.

The tablet according to the invention may have any desired shape, including, but not limited to, round concave, compound cup, round flat face, lozenge, modified ball, capsule, oval, ellipsoid, bullet, arrow head, triangle, square, pillow, rectangle, diamond, pentagon, hexagon, octagon, heart, half moon, almond, banana, and the like. Similarly, the tablet according to the invention may have any desired size. In specific embodiments, the tablet has a thickness less than about 20 mm, or, more specifically, a thickness of about 10 mm or less. In specific embodiments, the tablet may have a diameter or length and width dimensions in the range of about 2 to about 20 mm, more specifically from about 2 to about 10 mm. In one embodiment, the tablet has a disk configuration with a diameter of about 4 mm and a thickness of about 10 mm.

The following Examples demonstrate various aspects and improvements provided by the invention.

### Comparative Example 1

This Example demonstrates tablets prepared by direct compression using a conventional combination of tableting ingredients with a low melting point API comprising CBD (melting point 69-70° C.).

Approximately one kilogram of bulk tableting powder was prepared. The CBD was sieved through a 595 μm sieve. Flavored mannitol was created by adding 1 ml of methyl salicylate (wintergreen flavor, LorAnn Oils) to 50 grams of tableting mannitol (Pearlitol®, Roquette) in a container and shaking the container to mix thoroughly, until the powder was homogenous in appearance with no clumps.

CBD (melting point 69-70° C.), 40 grams, was placed into a tumbler mixer with 760 grams of tableting mannitol (Pearlitol®) and allowed to mix for one hour, after which 100 grams of standard MCC (MCC Avicel PH 102), 40 grams of crospovidone (VivaPharm®, JRS Pharma), and 50 grams of the flavored mannitol were added to the mixer. The powder was mixed for one half hour. Finally, magnesium stearate (ChemSavers), 10 grams, was added, and the powder was mixed for a further half hour.

The powder was placed into the hopper of a single-station tablet press (Riva MiniPress MII) and tableted at 70 tablets/minute until the hopper was empty. Significant sticking was observed throughout the run, with capping of the tablet surface and pronounced inability of the tablets to slide down the tablet press outlet ramp.

### Comparative Example 2

This Example demonstrates tablets prepared by direct compression using a conventional combination of tableting ingredients with a low melting point API comprising THC in the form of a purified oil at room temperature.

Approximately one kilogram of bulk tableting powder was prepared. THC, 10 grams purified distillate oil, ˜85% potency, was combined in a manual mortar and pestle with 10 grams of soy lecithin granules to form a doughy paste. The mixture was filtered through a 595 μm sieve to remove any large clumps. Flavored mannitol was prepared as described in Comparative Example 1 with 1 ml of methyl salicylate and 50 grams of tableting mannitol.

The THC-lecithin paste was placed into a tumbler mixer with 730 grams of tableting mannitol (Pearlitol®) and allowed to mix for one hour. After one hour, 100 grams of standard MCC (MCC Avicel PH 102), 40 grams of crospovidone (VivaPharm®), and 50 grams of flavored mannitol were added to the mixer, and the powder was mixed for a further half hour. Finally, magnesium stearate (ChemSavers), 10 grams, was added, and the powder was mixed for a further half hour.

The powder was placed into the hopper of a single-station tablet press (Riva MiniPress MII) and tableted at 70 tablets/minute until the hopper was empty. Significant sticking was observed throughout the run, starting with blurring of the logo letters as shown in FIG. 2 and eventually leading to complete capping of the tablets.

### Example 3

This Example demonstrates a tablet prepared by direct compression with a composition according to the invention.

Approximately one kilogram of bulk tableting powder was prepared. THC, 10 grams of purified distillate oil, ˜85% potency, was heated in an oven at 148° C. until the viscosity of the sample was reduced (about 10 minutes). Mannitol (Pearlitol®, 10 g) was added and stirred into the molten THC to make a slurry. The mixture was placed back into the oven for another 10 minutes, with periodic stirring. This procedure was repeated until added mannitol totaled 180 grams. Heating was continued until absorption of the THC into the mannitol was observed to be complete while maintaining the particulate form of the mannitol. The mixture was removed from the oven and allowed to cool completely at room temperature for half an hour. The resulting product was a dry, free-flowing granulate, and the granulate was then ground into an extremely fine powder with an electric mortar and pestle. The fine powder was filtered through a 595 μm sieve to remove any large clumps.

Soy lecithin granules (Fearn, Vitamin Cottage) were ground using a standard kitchen blender for several seconds. The ground lecithin was sieved through a 595 μm sieve to remove any large clumps. Flavored mannitol was prepared as described in Example 1.

The composite THC-mannitol powder was placed into a tumbler mixer with 490 grams of tableting mannitol (Pearlitol®) and allowed to mix for one hour, after which 150 grams of very small particle MCC (VivaPur®, JRS Pharma), 50 grams of SMCC (ProSolv®, JRS Pharma), 40 grams of crospovidone (VivaPharm®), 10 grams ground lecithin powder (Fearn), and 50 grams of flavored mannitol were added to the mixer, and the powder was mixed for a further half hour. Finally, sodium stearyl fumarate (Pruv®, JRS Pharma), 20 grams, was added, and the powder was mixed for a further half hour.

The powder was placed into the hopper of a single-station tablet press (Riva MiniPress MII) and tableted at 70 tablets/minute until the hopper was empty. No observable sticking was seen at any point throughout the run, the logo lines appeared crisp, and the tablet surface was shiny as shown in FIG. 3.

### Example 4

This Example demonstrates a tablet prepared by direct compression with a composition according to the invention.

THC, 2 grams of purified distillate oil, ˜85% potency, was heated in an oven at 148° C. until the viscosity of the sample was reduced (about 10 minutes). Mannitol (Pearlitol®, 10 g) was added and stirred into the molten THC to make a slurry. The mixture was placed back into the oven for another 10 minutes, with periodic stirring. This procedure was repeated until a total of 36 grams of mannitol was added. Heating was continued until absorption of the THC into the mannitol was observed to be complete. The mixture was removed from the oven and allowed to cool completely at room temperature for half an hour, and then ground into an extremely fine powder with an electric mortar and pestle. The mixture was filtered through a 595 μm sieve to remove any large clumps.

CBD was sieved through a 595 μm sieve. Soy lecithin granules (Fearn) were ground using a standard kitchen blender for several seconds. The ground lecithin was sieved through a 595 μm sieve to remove any large clumps. Flavored mannitol was prepared as described in Example 1.

The composite THC-mannitol powder was placed into a tumbler mixer with 582 grams of tableting mannitol (Pearlitol®) and 60 grams of CBD and allowed to mix for one hour. After one hour 100 grams of very small particle MCC (VivaPur®), 100 grams of SMCC (ProSolv®), 40 grams of crospovidone (VivaPharm®), 10 grams ground lecithin powder (Fearn), and 50 grams of flavored mannitol were added to the mixer, and the powder was mixed for a further half hour. Finally, sodium stearyl fumarate (Pruv®), 20 grams, was added, and the powder was mixed for a further half hour.

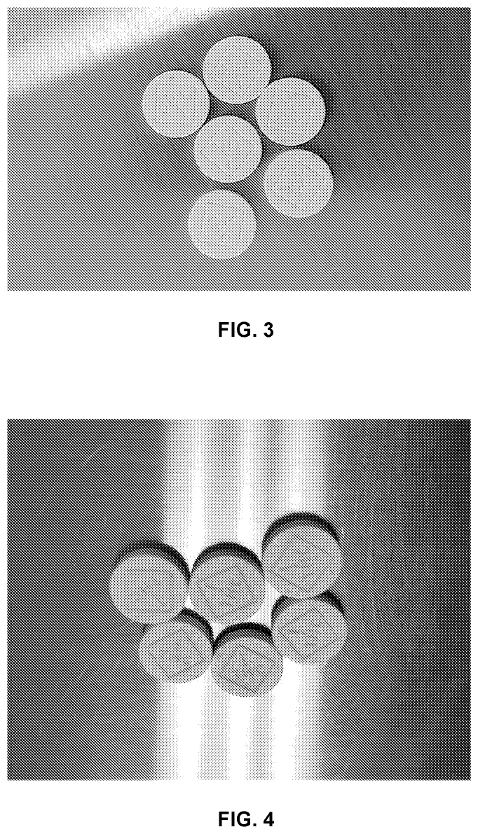

The powder was placed into the hopper of a single-station tablet press (Riva MiniPress MII) and tableted at 70 tablets/minute until the hopper was empty. No observable sticking was seen at any point throughout the run, the logo lines appeared crisp, and the tablet surface was shiny as shown in FIG. 4.

### Comparative Example 5

This Example demonstrates tablets prepared by direct compression using compositions which omit an important feature of the invention.

Specifically, each of the comparative compositions A-D as described in Table 1 (ingredients shown in parts by weight (pbw)) was prepared generally according to the procedure of Example 4, with the following exceptions:

Composition A was prepared without a granulation step and, instead, the TCH was heated in an oven at 148° C. until the viscosity of the sample was reduced (about 10 minutes).

Soy lecithin granules, 0.25 grams, were added to the molten THC and the slurry was ground into a tacky paste. Mannitol, 10 grams, was added, and the mixture was stirred manually in a mortar and pestle and formed a dough-like mass. The mixture was stirred until it appeared dry and was then passed through a sieve to produce a particulate form.

Composition B Omitted Lecithin.

Composition C replaced the small-sized microcrystalline cellulose with additional SMCC.

Composition D used magnesium stearate in place of sodium stearyl fumarate.

| | A | B | C | D |
|---|---|---|---|---|
| Ingredient | (pbw) | (pbw) | (pbw) | (pbw) |
| THC | 0.25 | 0.25 | 0.25 | 0.25 |
| Lecithin | 0.25 | — | 1.25 | 1.25 |
| Mannitol | 79 | 83.5 | 83.5 | 83.5 |
| CBD | 7.5 | 7.5 | 7.5 | 7.5 |
| MCC, <25 μm | 12.5 | 12.5 | — | 12.5 |
| SMCC | 12.5 | 12.5 | 25 | 12.5 |
| Crospovidone | 5 | 5 | 5 | 5 |
| sodium stearyl | 2.5 SSF | 2.5 SSF | 2.5 SSF | 2.5 MS |
| fumarate (SSF) or | | | | |
| Magnesium | | | | |
| stearate (MS) | | | | |

In each case, the mixed powder was placed into the hopper of a single-station tablet press (Riva MiniPress MII) and tableted at 70 tablets/minute until the hopper was empty.

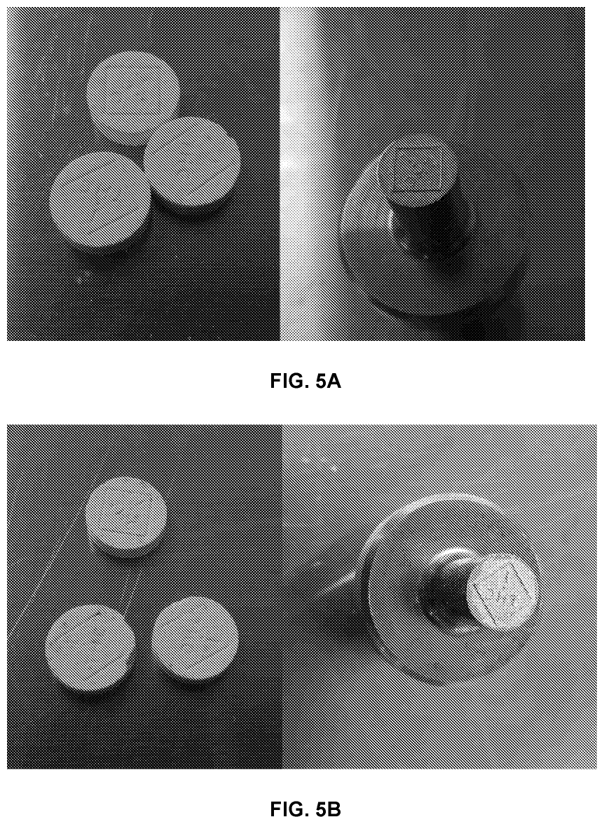

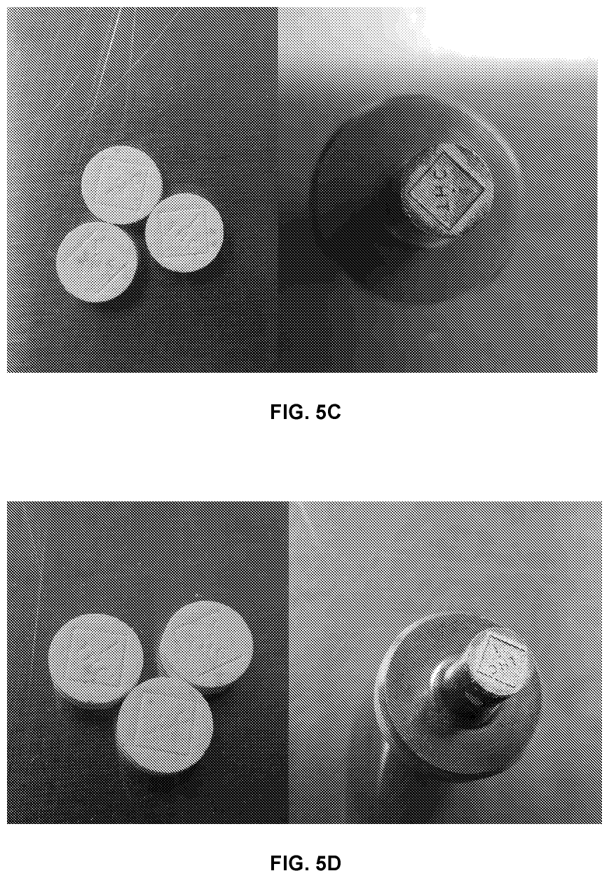

Tablets formed with composition A and the compression punch are shown in FIG. 5A. Despite the low number of presses (−400), signs of sticking were observed: the logos were ill-defined, and powder accumulation was observed on the surface of the punch, which is an early indicator of future tablet sticking.

Tablets formed with composition B and the compression punch are shown in FIG. 5B. Despite the low number of presses (−450), signs of sticking were observed: the logos were ill-defined, and powder accumulation was observed on the surface of the punch, which, as noted, is an early indicator of future tablet sticking.

Tablets formed with composition C and the compression punch are shown in FIG. 5C. Despite the low number of presses (−200), signs of sticking were observed: the logos were ill-defined, and powder accumulation was observed on the surface of the punch.

Tablets formed with composition D and the compression punch are shown in FIG. 5D. Despite the low number of presses (−250), signs of sticking were observed: the logos were ill-defined, and powder accumulation was observed on the surface of the punch.

These comparative compositions demonstrate the novel combination of elements of the inventive tablets provide the improved ability to efficiently form tablets containing low melting point actives by direct compression.

### Example 6

This Example demonstrates additional tablets prepared by direct compression with compositions E-H according to the invention.

In the preparation of each composition, THC, 0.25 grams of purified distillate oil, ˜85% potency, was heated in an oven at 148° C. until the viscosity of the sample was reduced (about 10 minutes). A granulation sugar/sugar alcohol (1.25 g) was added and stirred into the molten THC to make a slurry. The mixture was placed back into the oven for another 10 minutes, with periodic stirring. This procedure was repeated until the total sugar/sugar alcohol addition was 4.5 g. Heating was continued until absorption of the THC into the sugar/sugar alcohol was observed to be complete. The mixture was removed from the oven and allowed to cool completely at room temperature for half an hour, and then ground into a fine powder with an electric mortar and pestle. The mixture was filtered through a 595 μm sieve to remove any large clumps. CBD was sieved through a 595 μm sieve.

Flavored mannitol was created by adding 0.125 ml of methyl salicylate (wintergreen flavor) to 6.25 grams of tableting mannitol in a container and shaking the container to mix thoroughly, until the powder was homogenous in appearance with no clumps. Soy lecithin granules were ground using a standard kitchen blender for several seconds. The ground lecithin was sieved through a 595 μm sieve to remove any large clumps.

The composite THC-mannitol powder was placed into a tumbler mixer with 72.75 grams of tableting sugar/sugar alcohol and 7.5 grams of CBD and allowed to mix for one hour. After one hour, 12.5 grams of fine particle MCC (particle size less than 25 μm), 12.5 grams of SMCC, 5 grams of crospovidone, 1.25 grams ground lecithin powder, and 6.25 grams of flavored mannitol were added to the mixer, and the powder was mixed for a further half hour. Finally, 2.5 grams of sodium stearyl fumarate were added, and the powder was mixed for a further half hour.

Each powder composition was placed into the hopper of a single-station tablet press (Riva MiniPress MII) and tableted at 70 tablets/minute until the hopper was empty.

The granulation sugar/sugar alcohol and the tableting sugar/sugar alcohol in each composition were as follows:

| Composition | E | F | G | H |
|---|---|---|---|---|
| Granulation | Mannitol | Mannitol | Sucrose | Sorbitol |
| Tableting | Sucrose | Sorbitol | Mannitol | Mannitol |

For each of compositions E-H, no signs of sticking were observed during the direct compression process. The logos of the tablets appeared crisp and the tablet surface was shiny. The surface of the punch was clean with no signs of powder accumulation.

While the present invention has been illustrated by the description of embodiments and examples thereof, and while the embodiments have been described in considerable detail, it is not the intention of the Applicant to restrict or in any way limit the scope of the appended claims to such detail. Additional advantages and modifications will readily appear to those skilled in the art. Therefore, the application, in its broader aspects, is not limited to the specific details and illustrative examples shown and described. Accordingly, departures may be made from such details without departing from the spirit or scope of the invention defined by the claims.

1. A tablet, comprising a granulate of an active pharmaceutical ingredient comprising at least one cannabinoid having a melting point less than about 80° C.; sugar, sugar alcohol, or a combination thereof; microcrystalline cellulose having an average particle size less than about 25 μm; silica, silicified microcrystalline cellulose, or a combination thereof; and lubricant comprising sodium stearyl fumarate and lecithin.

2. The tablet of claim 1, wherein the active pharmaceutical ingredient has a melting point less than about 50° C.

3. The tablet of claim 1, wherein the active pharmaceutical ingredient has a melting point less than about 25° C.

4. The tablet of claim 1, wherein the active pharmaceutical ingredient comprises Δ9-tetrahydrocannabinol, cannabidiol, or a combination thereof.

5. The tablet of claim 1, wherein the granulate of the active pharmaceutical ingredient comprises at least a portion of the sugar, sugar alcohol, or combination thereof.

6. The tablet of claim 1, comprising from about 0.1 to about 20 wt % cannabinoid.

7. The tablet of claim 6, comprising from about 0.2 to about 10 wt % cannabinoid.

8. The tablet of claim 1, comprising from about 45 to about 80 wt % sugar, sugar alcohol, or combination thereof.

9. The tablet of claim 1, comprising from about 5 to about 25 wt % microcrystalline cellulose having an average particle size less than about 25 μm.

10. The tablet of claim 1, comprising from about 1 to about 20 wt % silica, silicified microcrystalline cellulose, or combination thereof.

11. The tablet of claim 1, comprising from about 0.5 to about 5 wt % of lubricant comprising sodium stearyl fumarate and lecithin.

12. The tablet of claim 1, wherein the lubricant comprises at least 50 wt % sodium stearyl fumarate, based on the combined weight of sodium stearyl fumarate and lecithin.

13. The tablet of claim 1, comprising from about 1 to about 10 wt % cannabinoid; from about 55 to about 75 wt % of the sugar, sugar alcohol, or combination thereof, from about 5 to about 20 wt % of the microcrystalline cellulose having an average particle size less than about 25 μm; from about 1 to about 15 wt % of the silica, silicified microcrystalline cellulose, or combination thereof, and from about 1 to about 4 wt % of the lubricant comprising sodium stearyl fumarate and lecithin, wherein the lubricant comprises at least 50 wt % sodium stearyl fumarate, based on the combined weight of sodium stearyl fumarate and lecithin.

14. The tablet of claim 1, further comprising from about 1 to about 10 wt % of crospovidone.

15. The tablet of claim 1, wherein the granulate of active pharmaceutical ingredient is formed by wet granulation.

16. The tablet of claim 1, wherein the granulate of active pharmaceutical ingredient is formed by melt granulation.

17. The tablet of claim 1, formed by direct compression.

18. A method of forming a tablet containing an active pharmaceutical ingredient having a melting point less than about 80° C., the method comprising
granulating a mixture of the active pharmaceutical ingredient comprising at least one cannabinoid, and sugar, sugar alcohol or a combination thereof,
mixing the resulting granulate with (a) additional sugar, sugar alcohol, or a combination thereof, (b) microcrystalline cellulose having an average particle size less than about 25 μm, (c) silica, silicified microcrystalline cellulose, or a combination thereof, and (d) lubricant comprising sodium stearyl fumarate and lecithin to provide a tablet formulation, and
direct compressing the resulting mixture to form a tablet.

19. The method of claim 18, wherein the granulating step comprises melt granulation.

20. The method of claim 18, wherein the granulating step comprises wet granulation.
